SEQUENCE LISTING (8 sequences)

SEQ ID NO: 1 - protein, 189 aa, Artificial Sequence
  mGM-CSF-mVEGF
SPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKVPGVGVPGVGNSDSECPLSHDGYCLHDGVCMYIEALDKYACNCVVGYIGERCQYRDLKWWELRHHHHHH

SEQ ID NO: 2 - protein, 256 aa, Artificial Sequence
  mGM-CSF-mVEGF
SPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKVPGVGVPGVGAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPEKCDKPRRHHHHHH

SEQ ID NO: 3 - protein, 128 aa, Artificial Sequence
  Recombinant human GM-CSF
MAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 4 - protein, 120 aa, Artificial Sequence
  mGM-CSF
SPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 5 - protein, 5 aa, Artificial Sequence
  linker peptide
VPGVG

SEQ ID NO: 6 - protein, 5 aa, Artificial Sequence
  linker peptide
GGGGS

SEQ ID NO: 7 - protein, 5 aa, Artificial Sequence
  linker peptide
PAPAP

SEQ ID NO: 8 - protein, 5 aa, Artificial Sequence
  linker peptide
EAAAK

(19) United States
(12) Patent Application Publication
(10) Pub. No.: US 2020/0308241 A1
(43) Pub. Date: Oct. 1, 2020

(54) NOVEL CONJUGATES AND USES THEREOF

(71) Applicants: Keng-Li LAN, Taipei (TW); Cheng-Liang TSAI, New Taipei City (TW)

(72) Inventors: Keng-Li LAN, Taipei (TW); Cheng-Liang TSAI, New Taipei City (TW)

(21) Appl. No.: 16/650,632

(22) PCT Filed: Sep. 27, 2018
(86) PCT No.: PCT/CN2018/107800

§ 371 Date: Mar. 25, 2020

Related U.S. Application Data
(60) Provisional application No. 62/565,509, filed on Sep. 29, 2017.

(51) Int. Cl. C07K 14/535; A61P 35/00; C07K 14/50; C07K 14/49; C07K 14/485

(52) U.S. Cl. CPC C07K 14/535; A61P 35/00; A61K 38/00; C07K 14/49; C07K 14/485; C07K 14/50

(57) ABSTRACT

The present invention relates to novel conjugates and pharmaceutical compositions comprising the novel conjugate described herein. The use of the novel conjugate to inhibit the growth of cancer cells and induce antibody production in a subject in need of cancer treatment, with or without an anti-cancer agent, is also provided.

### CROSS-REFERENCES TO RELATED APPLICATION

This application claims the benefit of priority from U.S. Provisional Application No. 62/565,509, filed 29 Sep. 2017, the entire content of which is incorporated herein by reference.

### BACKGROUND OF THE INVENTION

Epidermal growth factor receptor (EGFR) is a member of the ErbB family of tyrosine kinase receptors that transmit a growth-inducing signal to cells that have been stimulated by an epidermal growth factor (EGF) ligand. It is a key factor in epithelial malignancies, which accounts for more than 50% of all cancer cell death. EGFR activity enhances tumor growth, invasion, and metastasis, due to the sustained production of EGFs in the tumor microenvironment or as a result of a mutation in EGFR itself that locks the receptor in a state of continual activation.

EGFR antagonist and tyrosine kinase inhibitor are approved by the US Food and Drug Administration for the treatment of epithelial cancer. Despite the commercial success of EGFR antagonist, its use for the treatment of cancer is characterized by the development resistance through mutation of tyrosine kinase domain or upregulation of other oncoproteins.

To effectively treat cancer, it would be preferred if the body's initiate a positive immune response against these cancers. Granulocyte-macrophage colony-stimulating factor (GM-CSF) is as a cytokine that activates dendritic cells for antigen presentation and potentiates T- and B-lymphocyte antitumor functions. However, studies are conflicting on the role of GM-CSF as an immune potentiator or therapeutic agent as clinical evidence shows that GM-CSF may induce negative regulatory immune responses. For example, GM-CSF administered with a heat shock protein vaccine has been implicated in the induction of myeloid-derived suppressor cells in melanoma patients (Filipazzi P et al., Identification of a new subset of myeloid suppressor cells in peripheral blood of melanoma patients with modulation by a granulocyte-macrophage colony-stimulation factor-based antitumor vaccine J Clin Oncol 2007;25:2546-53). GM-CSF at high dose, in murine models, may increase myeloid-derived suppressor cells (Serafini et al., High dose granulocyte-macrophage colony-stimulating factor-producing vaccines impair the immune response through the recruitment of myeloid suppressor cells. Cancer Res 2004;64:6337-43). A randomized multicenter study to address the role of GM-CSF administered locally as a vaccine adjuvant show that the circulating T-cell response to the multipeptide vaccine is significantly lower in patients whose vaccines included GM-CSF. Reasons for this remain to be defined, but the findings cast doubt on the benefit of GM-CSF protein as local adjuvant. (CL Slingluff Jr et al., Effect of granulocyte/macrophage colony-stimulating factor on circulating CD8+ and CD4+ T-cell responses to a multipeptide melanoma vaccine: outcome of a multicenter randomized trial. Clin Cancer Res. 2009;15(22):7036-7044.)

Accordingly, there is a need for a more effective immune based cancer therapy. The present invention addresses this need and other needs.

### BRIEF SUMMARY OF THE INVENTION

Embodiments of the present invention provide conjugates comprising a granulocyte macrophage colony stimulating factor (GM-CSF) polypeptide; and a ligand.

The present invention also provides pharmaceutical compositions, comprising the conjugate described herein and a pharmaceutically acceptable excipient.

According to one embodiment of the present invention, methods are provided for inhibiting cancer cell growth in a subject. The method comprises administering an effective amount of the conjugate described herein, wherein said effective amount of the conjugate inhibit cancer cell growth in said subjects.

According to another embodiment of the present invention, methods for inducing anti-EGF antibody in a subject are provided. The method comprising the step of administering an effective amount of a conjugate comprising GM-CSF polypeptide and EGF in the subject in need of cancer treatment, wherein an effective amount of anti-EGF antibody is induced in said subject.

According to yet another embodiment of the present invention, methods for inducing anti-vascular endothelial growth factor (VEGF) antibody in a subject is provided. The method comprising the step of administering an effective amount of a conjugate comprising GM-CSF polypeptide and VEGF to the subject in need of cancer treatment, wherein an effective amount of anti-VEGF antibody is induced in said subject.

Also provided are the use of the conjugate described herein in the manufacture of a medicament for therapeutic and/or prophylactic treatment of cancer.

Further provided is a therapeutic agent for inhibiting cancer cells, comprising a therapeutically effective amount of the conjugate described herein.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

A further understanding of the nature and advantages of the present invention may be realized by reference to the remaining portions of the specification and the drawings.

### BRIEF DESCRIPTION OF THE DRAWINGS

The patent or patent application contains at least one drawing executed in color. Copies of this patent or patent application with color drawings will be provided by the Office upon request and payment of the necessary fee.

FIG. 1A shows the amino acid sequence of one embodiment of the conjugate, mGM-CSF-hEGF (SEQ ID NO:1) of the present invention. FIG. 1B shows the amino acid sequence of another embodiment of the conjugate, mGM-CSF-mVEGFa (SEQ ID NO:2) of the present invention. FIG. 1C shows the amino acid sequence of the third embodiment of the conjugate, mGM-CSF-mPDGFA (SEQ ID NO:3) of the present invention. FIG. 1D shows the amino acid sequence of the fourth embodiment of the conjugate, mGM-CSF-mbFGF (SEQ ID NO:4) of the present invention.

FIG. 2A and FIG. 2B illustrate schematically the mechanisms of action of the conjugate of the present invention.

FIG. 3 is a chromatography image illustrating the purification of mGM-CSF-hEGF, using nickel ion column.

FIG. 4 is an SDS-PAGE and Cootnass e Brilliant Blue staining of the purified proteins at different purification steps.

FIG. 5 is line graph illustrating the tumor size in mice injected with (a) murine renal cell carcinoma (RENCA) cells only (Parental), (b) RENCA cells mixed with cells expressing mGM-CSF (mGM-CSF), (c) RENCA cells mixed with cells expressing mGM-CSF-hEGF (mGM-CSF-hEGF) or (d) RENCA cells mixed with cells expressing mGM-CSF-mVEGF (mGM-CSF-mVEGF).

FIG. 6A is a photographic image illustrating the tumor burden in mice treated with PBS buffer or GM-CSF-EGF conjugate. FIG. 6B is a bar graph illustrating the bioluminescent intensity from luciferase in mice treated with PBS buffer or GM-CSF-EGF conjugate.

FIG. 7A and FIG. 7B are fluorescence activated cell sorting (FACS) images illustrating the binding of GM-CSF-EGF conjugate of Example 1, EGF and PBS to EGF receptors on breast cancer cells (MDA468) and renal cell carcinoma (RENCA), respectively.

FIG. 8 shows the effect of mGM-CSF-EGF on GM-CSF dependent NFS60 leukemia cell proliferation.

FIG. 9 is line graph illustrating the binding affinity of hEGF and GM-CSF-hEGF conjugate of Example 1 to EGF receptors.

FIG. 10A is a line graph showing the anti-EGF antibody titer in mice injected with GM-CSF-EGF conjugate of Example 1 and PBS. FIG. 10B is a line graph showing the serum of mGM-CSF-EGF immunized mice suppresses the binding of EGF to EGF receptors.

FIG. 11 is a line graph showing the tumor size in the 4 groups of mice inoculated with the B16-F10 cells expressing mGM-CSF or mGM-CSF-hEGF, with or without an anti-CTLA-4 antibody.

FIG. 12 is a line graph illustrating the tumor size in mice injected with B16-F10 cells expressing mGM-CSF, mGM-CSF-mPDGFA and mGM-CSF-mbFGF respectively.

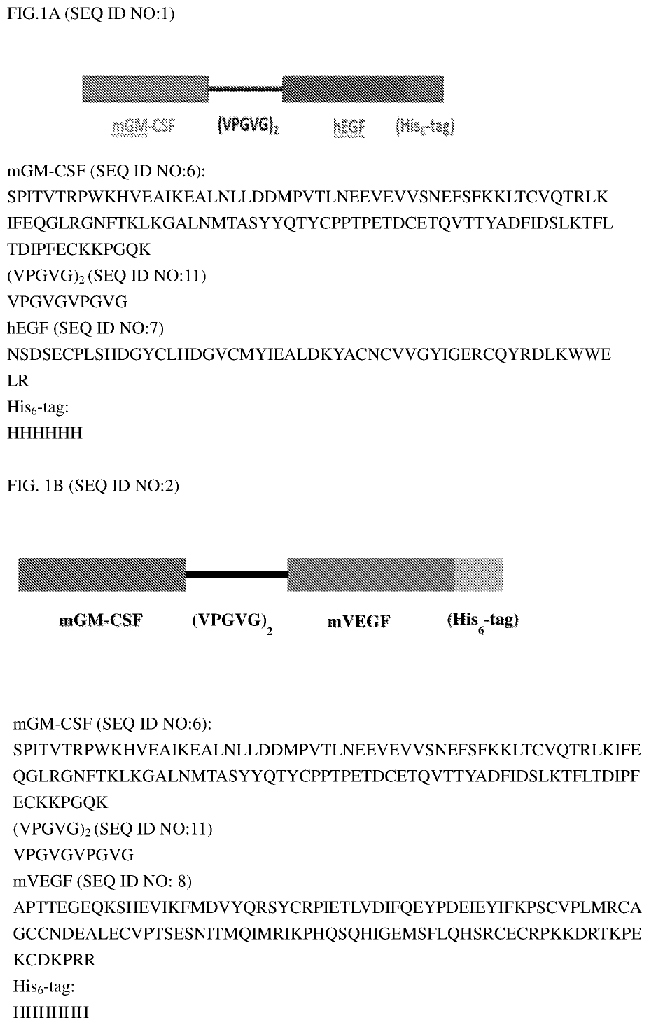

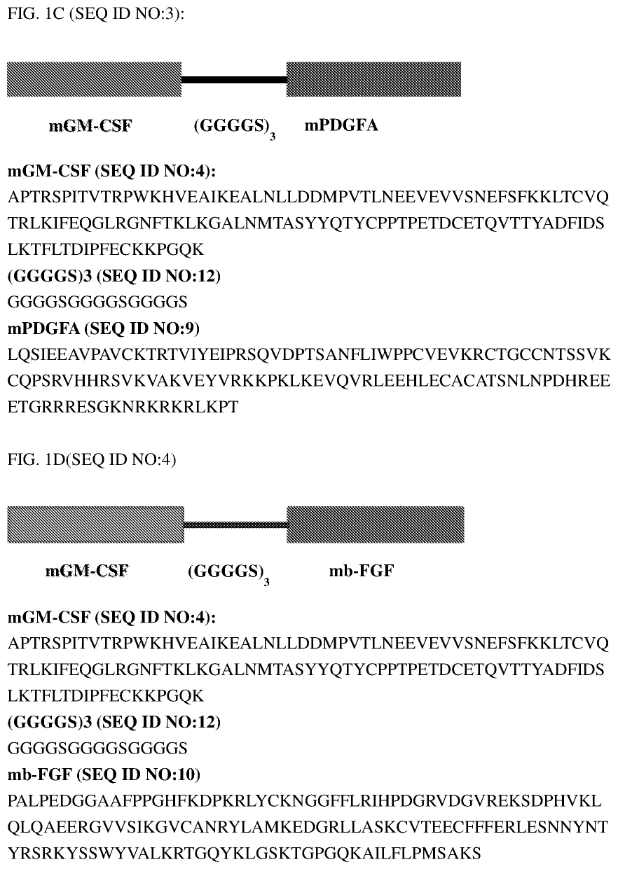

### DETAILED DESCRIPTION OF THE INVENTION

As used herein, the articles “a” and “an” refer to one or more than one (i.e., at least one) of the grammatical object of the article. By way of example, “an element” means one element or more than one element.

An “effective amount,” as used herein, refers to a dose of the conjugate to inhibit cancer cells or that is sufficient to reduce the symptoms and signs of cancer, such as weight loss, pain and palpable mass, which is detectable, either clinically as a palpable mass or radiologically through various imaging means. The term “effective amount” and “therapeutically effective amount” are used interchangeably.

The term “subject” can refer to a vertebrate having cancer or to a vertebrate deemed to be in need of cancer treatment. Subjects include all warm-blooded animals, such as mammals, such as a primate, and, more preferably, a human. Non-human primates are subjects as well.

The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

All numbers herein may be understood as modified by “about.” As used herein, the term “about,” when referring to a measurable value, is meant to encompass variations of ±10% from the specified value, as such variations are appropriate.

### Conjugates

The present invention provides a conjugate or a fusion protein, comprising a granulocyte macrophage colony stimulating factor (GM-CSF) polypeptide; and a ligand. In some embodiment, the conjugate or the fusion protein further comprises a linker to link the GM-CSF polypeptide to the ligand to form the conjugate.

GM-CSF polypeptide refers to a family of glycoprotein growth factors that control the production, differentiation, and function of granulocytes and monocytes-macrophages. GM-CSF polypeptide is produced by a number of different cells, such as activated T cells, B cells, macrophages and mast cells, in response to cytokine, immune and inflammatory stimuli. Recombinant GM-CSF is a glycoprotein of various amino acids and can have a variety of molecular weights depending on the extent of glycosylation. Exemplary, but by no means the only form of such molecules, can be seen in U.S. Pat. No. 5,602,007 and U.S. Pat. No. 5,891,429, incorporated by reference. In one embodiment, the GM-CSF is at least 90%, 91%, 92%, 93%, 94%, 95%, 96%, 97%, 98%, 99% or 100% homologous to a SEQ ID NO:5 (the recombinant GM-CSF). In another embodiment, the GM-CSF is at least 90%, 91%, 92%, 93%, 94%, 95%, 96%, 97%, 98%, 99% or 100% homologous to a SEQ ID NO:6 (the signaling sequence of GM-CSF).

In one embodiment, the ligand is polypeptide. In another embodiment, the ligand is a tumor associated ligand, which stimulates cancer cell growth. In another embodiment, the ligand is a non-antibody ligand, i.e., the ligand is not an antibody. In yet another embodiment, the ligand is a non-interleukin ligand, i.e., the ligand is not an interleukin, In yet another embodiment, the ligand is not a receptor expressed by a cancer cell.

In certain embodiments, the tumor associated ligand directly or indirectly interacts with the surface of a tumor cell, a macrophage or an endothelial cell. In certain embodiments, the tumor associated ligand is affinitive to a tumor cell receptor, a macrophage receptor, or an endothelial cell receptor.

Non limiting examples of the tumor associated ligand that directly interacts with the tumor cell surface or is affinitive to the tumor cell receptor are epidermal growth factor (EGF) polypeptide (for example, SEQ ID NO:7), C-X-C motif chemokine 12 (CXCL12) polypeptide, Hepatocyte Growth Factor-1 (HGF) polypeptide, Insulin-like Growth Factor (IGF) polypeptide, transforming growth factor-α (TGF-α), or an active fragment or variant thereof. Non limiting examples of the tumor associated ligand that directly interacts with the endothelial cell surface or is affinitive to the endothelial cell receptor are vascular endothelial growth factor (VEGF) polypeptide (for example, SEQ ID NO: 8), Platelet Derived Growth Factor (PDGF) polypeptide (for example, SEQ ID NO:9), Fibroblast growth factor (FGF) polypeptide (for example, SEQ ID NO:10) or an active fragment or variant thereof. Non limiting examples of the tumor associated ligand that directly interacts with the macrophage surface or is affinitive to the macrophage receptor are colony stimulating factor (CSF-1) polypeptide, macrophage chemoattractant protein-I (MCP-1) polypeptide, macrophage inflammatory protein-1α (MIP-1α) polypeptide, or an active fragment or variant thereof.

In an exemplary embodiment, the conjugate is a fusion protein. In certain embodiment, the conjugate or the fusion protein comprises an amino acid sequence at least 90%, 91%, 92%, 93%, 94%, 95%, 96%, 97%, 98%, 99% or 100% homologous to SEQ ID NO: 1, SEQ ID NO: 2, SEQ ID NO: 3 or SEQ ID NO:4.

Percent homology between amino acid can be determined using standard methods known to those of skill in the art. For instance for determining the percentage of homology between two amino acid sequences, the sequences are aligned for optimal comparison purposes. The amino acid residues at corresponding amino acid positions are then compared. When a. position in the first sequence is occupied by the same amino acid residue as the corresponding position in the second sequence, then the sequences are homologous or identical at that position. The percent homology between the two sequences is a function of the number of identical or homologous positions shared by the sequences, taking into account the number of gaps which need to be introduced for optimal alignment and the length of each gap. The comparison of sequences and determination of percent homology between two sequences are well known in the art. The NCBI Basic Local Alignment Search Tool (BLAST (Altschul et al, J. Mol. Biol. 215:403, 1990) is available from several sources, including the National Center for Biotechnology Information (NCBI, Bethesda, Md.) and on the internet, for use in connection with the sequence analysis programs, such as blastn. A description of how to determine sequence identity using this program is available on the NCBI website.

In some embodiments, the conjugate differs from SEQ ID NO:1, SEQ ID NO:2, SEQ ID NO:3 or SEQ ID NO:4 by a small number of functionally inconsequential amino acid substitutions (e.g., conservative substitutions), deletions, or insertions and retain the functional properties of SEQ ID NO:1, SEQ ID NO:2, SEQ ID NO:3 or SEQ ID NO:4, i.e., the conjugate inhibits the cancer cell growth in at least one, and preferably all, in vitro or in vivo assays described herein. For purposes of classifying amino acids substitutions as conservative or nonconservative, amino acids may be grouped as follows: Group I (hydrophobic side chains): norleucine, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another. Conservative amino acid substitution was known in the art. For example, P Ng et al. (Annu. Rev. Genomics Hum. Genet. 2006. 7:61-80) gives guidance on conservative amino acid modification while retaining protein stability and function: if tyrosines and tophans are present at a particular site, one would expect that the other aromatic amino acid, phenylalanine, would also be tolerated at that site.

In one embodiment, the conjugate differs from SEQ ID NO:1, SEQ ID NO:2, SEQ ID NO:3 or SEQ ID NO:4 by up to 5 amino acids change, such as 1, 2, 3, 4, or 5 amino acids change.

In certain embodiment, the conjugate is formed as a result of a covalent, non-covalent attachment or linkage of GM-CSF to a ligand. One example of a conjugate is a “fusion protein” or “fusion polypeptide,” that is, a polypeptide that is created through the joining of two or more coding sequences, which originally coded for separate polypeptides; translation of the joined coding sequences results in a single, fusion polypeptide. The conjugate can be produced recombinantly. In some embodiments, GM-CSF is operatively linked to the ligand by a linker to form the fusion protein. Non limiting examples of the linker peptide include (VPGVG)n (SEQ ID NO:11), any glycine-rich linker such as (GGGGS)n (SEQ ID NO:12) (PAPAP)n (SEQ ID NO:13) and (EAAAK)n (SEQ ID NO:14), wherein n is an integer between 2 to 4. A person skilled in the art can ascertain the most suitable linker peptide using methods known in the art.

Alternatively, GM-CSF and the ligand can be independently recombinantly produced and linked via chemical means to form the conjugate described herein.

In some embodiments, the conjugate of the present invention is administered with an anti-cancer agent. Non-limiting examples of anti-cancer agents can be found in Cancer Principles and Practice of Oncology by V. T. Devita, T. Lawrence and S. Rosenberg (editors), 9^{th} edition (2011), Lippincott Williams & Wilkins Publishers. Non-limiting examples of such anti-cancer agents include: chemotherapy (e.g., alkylating agents, platinum analogs, anti-metabolites), targeted therapy which inhibit the growth of cancer cells by interfering with specific targeted molecules needed for carcinogenesis and cancer growth, rather than by simply interfering with rapidly dividing cells (e.g., with conventional chemotherapeutic agent). In some embodiments, the target cancer therapy comprises kinase inhibitor, angiogenesis inhibitor, epidermal growth factor receptor (EGFR) inhibitor, HER2/neu receptor or the combination thereof, surgery, radiotherapy, biotherapeutics (e.g., interleukin therapy, gene therapy, cancer vaccine, antibody therapy, immunotherapy), or a combination thereof.

In one embodiment, the anti-cancer agent is Cytotoxic T-lymphocyte-associated antigen 4 (CTLA-4) antibody. In another embodiment, the anti-cancer agent is an EGFR antibody. In yet another embodiment, the anti-cancer agent is a tyrosine kinase inhibitor. Without being bound by any particular theory, it is believed that the combination of the conjugate of the present invention and an anti-cancer agent, such as an CTLA-4 antibody, an EGFR antibody, or a tyrosine kinase inhibitor is synergistic in cancer cell inhibition, wherein one or even all of the lower dosages of the anti-cancer agents would not be sufficient to have a therapeutic effect when the respective anti-cancer agent is used in monotherapy.

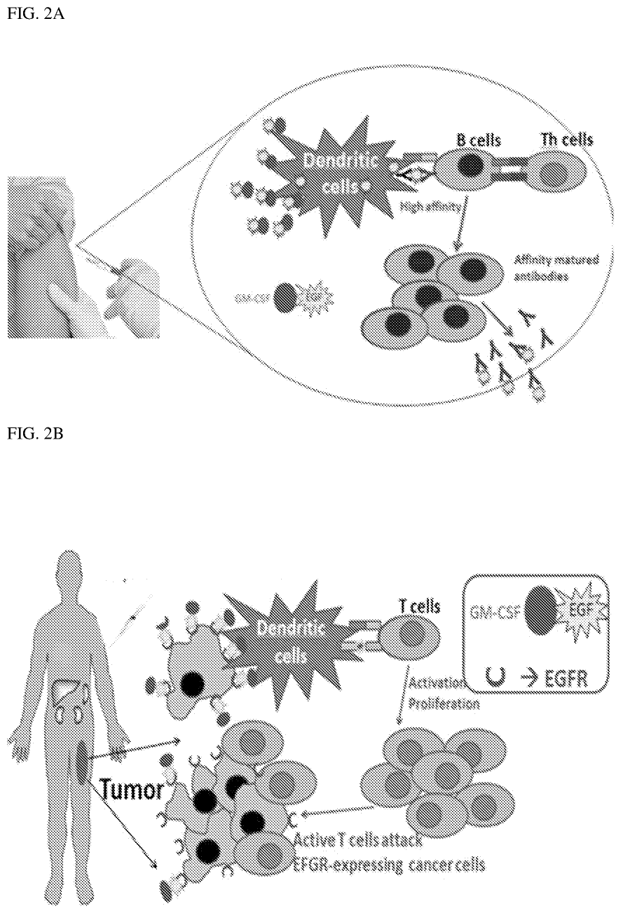

FIG. 2A and FIG. 2B illustrate the mechanisms of action of the conjugate of the present invention, by priming the immune system to attack cancer cells. In one embodiment, as illustrated in FIG. 2A, the conjugate of the present invention (GM-CSF/EGF) binds to the dendritic cell and the GM-CSF of the conjugate acts as a potent adjuvant to elicit the production of the ligand (EGF) specific antibody. In other embodiment, as illustrated in FIG. 2B, the ligand of the conjugate interacts with the cancer cell surface or is affinitive to the cancer cell receptor (for example, EGF ligand binds with EGFR of the cancer cell). This triggers the dendritic cells and subsequently activates anti-cancer T cells. The activated T cells attack the EGFR expressing cancer cells.

### Pharmaceutical Composition

The invention also provides pharmaceutical compositions, comprising the conjugate described herein and a pharmaceutically acceptable vehicle, excipient or carrier. In certain embodiment, the pharmaceutical composition further comprises an anti-cancer agent. In certain embodiment, the pharmaceutical composition comprises about 80%, about 85%, about 90%, about 95%, about 99%, about 99.5% or about 80-99.5% of the conjugate and about 0.5%, about 1%, about 5%, about 10%, about 15%, about 20% or about 0.5-20% of the conjugate.

Suitable vehicles are, for example, water, saline, dextrose, glycerol, ethanol, or the like, and combinations thereof. In addition, the vehicle can contain other excipients, such as wetting or emulsifying agents, pH buffering agents, or adjuvants. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, dextrans, antioxidants, such as ascorbic acid or glutathione, chelating agents, low molecular weight proteins, detergents, liposomal carriers, or other stabilizers and/or buffers. The excipients may be nonionic surfactants, polyvinylpyrollidone, human serum albumin, aluminum hydroxide, agents with anesthetic action, and various unmodified and derivatized cyclodextrins. More preferably, the nonionic surfactants may include Polysorbate 20, Polysorbate 40, Polysorbate 60, and Polysorbate 80. The polyvinylpyrollidone may preferably be Plasdone C15, a pharmaceutical grade of polyvinylpyrollidone. The agent having anesthetic action preferably is benzyl alcohol. Other physiologically acceptable compounds include wetting agents, emulsifying agents, dispersing agents or preservatives. See e.g., the 21st edition of Remington's Pharmaceutical Science, Mack Publishing Company, Easton, Pa. (“Remington's”). The pharmaceutical compositions of the present invention can also include ancillary substances, such as pharmacological agents or other biological response modifiers. The pharmaceutical composition comprising such excipient or carrier is formulated by well-known conventional methods.

The pharmaceutical composition may be formulated for the following route of administration: intravascular, intramuscular, oral, dermal, nasal, buccal, rectal, vaginal, by inhalation, or by subcutaneous administration. Other modes of administration may be applicable as long as a satisfactory immunogenicity can be induced.

The pharmaceutical compositions of the present invention can be prepared as injectables, either as liquid solutions or suspensions, or as solid forms which are suitable for solution or suspension in liquid vehicles prior to injection. The pharmaceutical composition can also be prepared in solid form, emulsified or the active ingredient encapsulated in liposome vehicles or other particulate carriers used for sustained delivery. For example, the pharmaceutical composition can be in the form of an oil emulsion, water-in-oil emulsion, water-in-oil-in-water emulsion, site-specific emulsion, long-residence emulsion, stickyemulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of the vaccine.

### Methods of Inhibiting Cancer Cell Growth

According to the present invention, methods for inhibiting cancer cell growth are provided by administering an effective amount of the conjugate described herein, comprising an GM-CSF polypeptide and a ligand, wherein said effective amount of the conjugate inhibit cancer cell growth in said subject.

The conjugate of the present invention can be administered at any effective amount. Useful dosages of the conjugate to suppress the cancer cells are determined by comparing their in vitro activity, and in vivo activity in animal models. Methods for the extrapolation of effective dosages in mice, and other animals, to humans are known in the art; for example, see U.S. Pat. No. 4,938,949, which is incorporated by reference herein. The dosage of the conjugate to suppress cancer cells will depend on the severity of the condition being treated, the particular formulation, and other clinical factors such as weight and the general condition of the recipient and route of administration.

In some embodiment, the method for inhibiting cancer cells further comprises administering an effective amount of an anti-cancer agent, wherein the anti-cancer agent is a biotherapeutics, chemotherapy, target therapy, surgery, radiotherapy, or a combination thereof.

In other embodiment, the method for inhibiting cancer cells comprises the administering the conjugate described herein and an anti-cancer agent selected from a CTLA-4 antibody, an EGFR antibody, a tyrosine kinase inhibitor, or a combination thereof to a subject in need thereof.

In one embodiment, the cancer cell expresses epidermal growth factor receptor (EGFR). Non limiting examples of EGFR expressing cancer are lung cancer, head and neck cancer, skin cancer, esophageal cancer, pancreatic cancer, stomach cancer, colorectal cancer, renal cell carcinoma, breast cancer, ovarian cancer, glioma, bladder cancer, hepatocellular carcinoma, prostate cancer or esophageal cancer. In another embodiment, the cancer cell expresses vascular endothelial growth factor (VEGF). Non limiting examples of VEGF expressing cancer are lung cancer, head and neck cancer, skin cancer, esophageal cancer, pancreatic cancer, stomach cancer, colorectal cancer, renal cell carcinoma, breast cancer, ovarian cancer, glioma, bladder cancer, hepatocellular carcinoma, prostate cancer or esophageal cancer.

The conjugate may be administered in a single dose treatment or in multiple dose treatments, over a period of time appropriate to the condition being treated. The conjugate may conveniently be administered at appropriate intervals, for example, once a day, twice a day, three times a day, once every second day, once every three days or once every week, once a month, one over a period of at least 3 months or until the symptoms and signs of the condition resolved.

### Methods for Inducing Antibody Production

Also disclosed are methods for inducing antibody production, wherein the antibody is effective against cancer cells.

In one embodiment, methods for inducing anti-EGF antibody are provided, comprising administering an effective amount of a conjugate comprising an GM-CSF polypeptide and an EGF ligand to a subject in need of cancer treatment, wherein an effective amount of anti-EGF antibody is induced in said subject.

In another embodiment, methods for inducing anti-VEGF antibody are provided, comprising administering an effective amount of a conjugate comprising an GM-CSF polypeptide and an VEGF ligand to a subject in need of cancer treatment, wherein an effective amount of anti-VEGF antibody is induced in said subject.

Embodiments of the present invention are illustrated by the following examples, which are not to e construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention. During the studies described in the following examples, conventional procedures were followed, unless otherwise stated.

### Example 1

### Expression of mGM-CSF-hEGF in E.coli

To construct the mGM-CSF-hEGF conjugate or fusion protein, GM-CSF and EGF were cloned into pET56 an E. coli. expression vector, followed by transforming the pET56 mGM-CSF-hEGF construct into the Rosetta (DE3) strain of E. coli.

E. coli was grown in 10 ml lysogeny broth (LB) culture containing 50 μg/ml of Ampicilin overnight at 37° C. The 10 ml LB culture was used to inoculate 1 liter of LB containing Ampicilin (in two 1 L Fernbach flask) the following morning. Prior studies of various culture conditions show that the addition of 0.03 mM isopropyl b-D-1-thiogalatopyranoside (IPTG) at a culture optical density (600 nm) of approximately 0.6 and subsequent culturing for 16 hours at 37° C. resulted is the most optimal condition to improve the yield of mGM-CSF-hEGF inclusion body. Cells were harvested by centrifugation at 5000 g at 4° C. for 15 minutes and stored at −20° C.

E. coli was re-suspended in 150 ml of re-suspend buffer PBS (134 mM NaCl, 2.7 mM KCl, Na₂HPO₄ 10 mM and KH₂PO₄ at pH 7.4). French Press mechanical disruption was used to lyse the bacteria. The inclusion bodies were isolated from the cell supernatant by centrifugation at 5000 g at 4° C. for 20 minutes. The inclusion body pellets were solubilized in PBS (by repeatedly passing the inclusion bodies through a dropper) and washed repeatedly using different washing buffers (1^{st} wash was with 0.1% SDC in PBS, 2^{nd} wash was with PBS, 3^{rd} wash was with PBS, and 4^{th} wash was with H₂O). The washed inclusion bodies were denatured in 20 ml denature buffer (50 mM Tris-base, 150 mM NaCl, 6 M Urea, at pH 8) at 60 rpm at 4° C. for 16 hours to form the denatured protein solution.

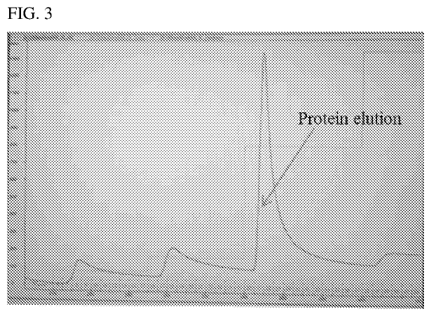

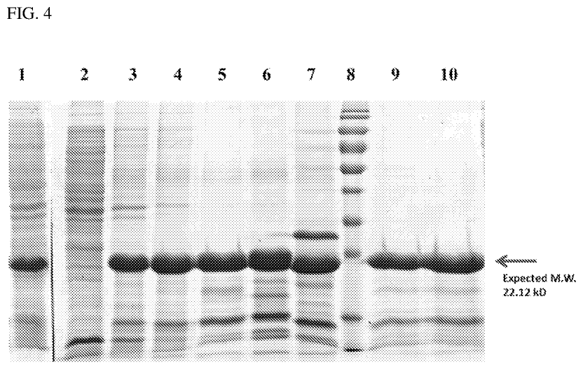

The denatured protein solution was clarified by centrifugation (13200 g at 4° C. for 20 minutes). The supernatant was loaded onto a HiTrap His-tag affinity column (GE Healthcare Life Sciences, USA) equilibrated in an equilibration buffer (50 mM Tris-base, 150 mM NaCl and 6M Urea, at pH 8). The column was washed with an excess amount of wash buffer (50 mM Tris-base, 150 mM NaCl and 6M Urea at pH 8). The mGM-CSF-hEGF was eluted by an elution buffer (50 mM Tris-base,150 mM NaCl, and 6M Urea) with an increased concentration of Immidazole (from 0-250 mM). FIG. 3 is the nickel-affinity chromatography image, with the proteins detected using UV light and the concentrations were indicated as blue line. To visualize the eluted protein, 5 μl of the protein fraction was checked for purity by SDS page gel stained with Coomassie Brilliant Blue for 20 min. FIG. 4 shows the purified protein products at difference purification step: 1. Initial bacteria lysate; 2. Supernatant of bacterial lysate; 3. Pellet; 4. Inclusion body; 5. Washed inclusion body; 6. Inclusion body dialysis; 7. Refolded solubilized inclusion body; 8. Molecular weight marker; 9. mGM-CSF-hEGF (SEQ ID NO:1) and 10. mGM-CSF-hEGF (SEQ ID NO:1).

### Example 2

### GM-CSF-EGF Fusion Protein Binding to EGFR

To examine the bioactivity of purified mGM-CSF-hEGF fusion protein of Example 1, murine myeloblastic leukemia cell line, NFS-60, was plated in the 96-well plate at 5000 cells/well and incubated with an increasing concentration of mGM-CSF-hEGF or mGM-CSF at 37° C. for 3 days. The viable NFS-60 cells were measured using the a colorimetric dimethylthiazol-2-yl)-2,5-diphenyltetrazolium bromide (MTT) assay. The purple formazan produced in viable NFS-60 cells by succinate dehydrogenase was detected at 540 nm using a ELISA plate reader. FIG. 8 shows mGM-CSF-EGF stimulates GM-CSF dependent NFS60 proliferation. GM-CSF-EGF fusion protein has an EC₅₀ of 20 pM in GM-CSF-dependent NSF60 proliferation assay, whereas the EC₅₀ of purified wild-type GM-CSF protein is 31 pM.

In the EGF-EGFR binding assay, both wild-type EGF and mGM-CSF-hEGF fusion protein of Example 1 display a high affinity with a Kd value of approximately 10 nM.

Binding of mGM-CSF-hEGF of Example 1 and hEGF to Purified EGFR

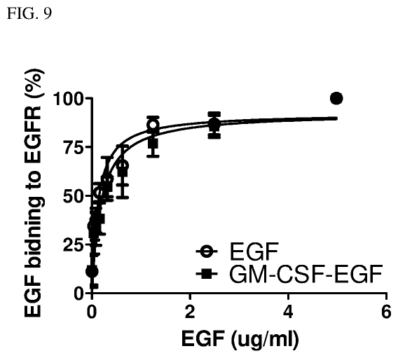

Purified EGFR (R&D Systems, Minneapolis, Minn.) was diluted in coating buffer (0.2 M sodium carbonate/bicarbonate pH 9.4, 0.5 1 g/mL) and immobilized on an ELISA plate by incubation at 4° C. overnight. Various concentrations of mGM-CSF-hEGF and hEGF (0-50 nM) were incubated with immobilized EGFR at room temperature for 1 h, followed by washing the ELISA plate three times with PBS buffer. The in vitro binding of either his6-tagged mGM-CSF-hEGF or hEGF protein with EGFR was detected using an HRP-tagged, anti-his6 antibody and developed by the addition of the HRP substrate (100 μL/well), 3,30,5,50-tetramethylbenzidine (TMB). The peroxidase reaction was stopped 30 min after the addition of 0.5 M H₂SO₄ (50 mL/well), and the absorbance was measured at 450 nm with a multichannel microtiter plate reader. As illustrated in FIG. 9, both wild-type hEGF and mGM-CSF-hEGF of Example 1 display a high affinity with a Kd value of less than 5nM in the EGF-EGFR binding assay.

### Example 3

### The Anti-Cancer Efficacy of the GM-CSF-EGF and GM-CSF-VEGF Fusion Proteins

An in vivo study was conducted to evaluate the anticancer efficacy of GM-CSF-EGF (SEQ ID NO:1) and GM-CSF-VEGF (SEQ ID NO:2) fusion proteins using c57 mice.

The mice were subcutaneously inoculated with (a) 2×10⁵ parental B16-F10 murine melanoma cells, (b) 1×10⁵ parental B16-F10 cells mixed with 1×10⁵ stable B16-F10 cells expressing mGM-CSF (mGM-CSF), (c) 1×10⁵ parental B16-F10 cells mixed with 1×10⁵ stable B16-F10 cells expressing mGM-CSF-hEGF (SEQ ID NO:1), or (d) 1×10⁵ parental B16-F10 cells mixed with 1×10⁵ stable B16-F10 cells expressing mGM-CSF-mVEGF (SEQ ID NO:2).

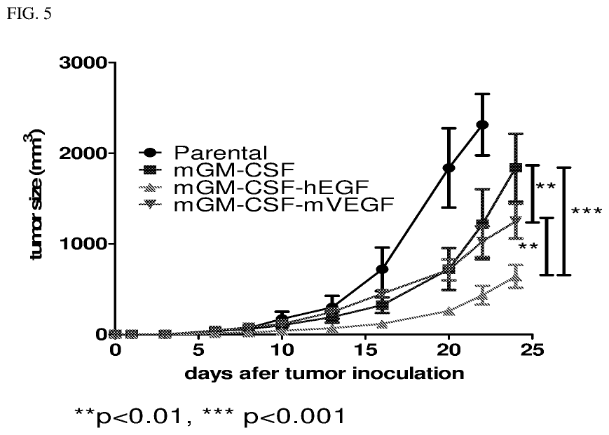

FIG. 5 shows the tumor size in the 4 groups of mice inoculated with or without the B16-F10 cells expressing mGM-CSF-hEGF and mGM-CSF-mVEGF. The mice inoculated with the B16-F10 cells expressing mGM-CSF-hEGF (group c) and mGM-CSF-mVEGF (group d) had significant tumor reduction compared to the mice inoculated with B16-F10 cells tumor expressing mGM-CSF only (group b).

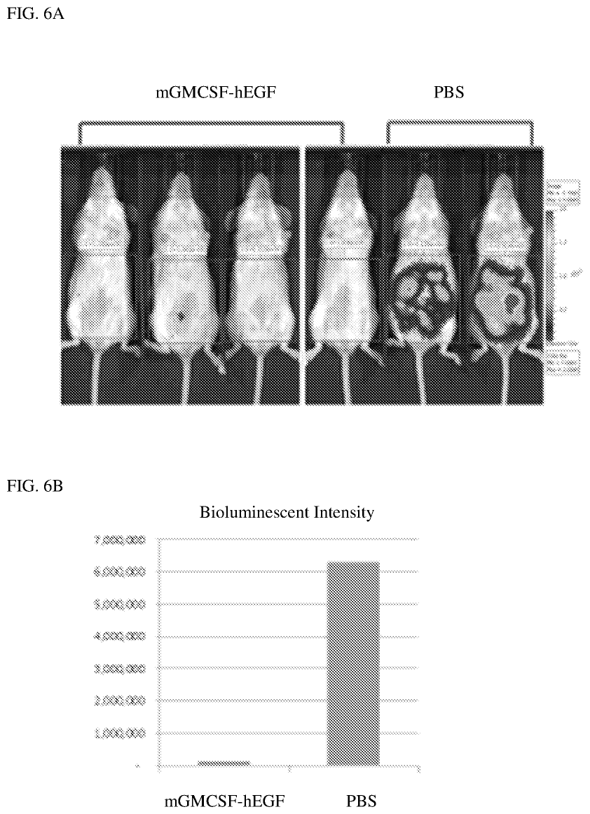

Additional animal study was conducted using Balb/c mice. The mice were intraperitoneally (i.p.) inoculated with RENCA-hEGFR-luc tumor cells, which express both hEGFR and luciferase. On the same day of the tumor inoculation, the mice were administered with daily injection of PBS buffer (the control group) or daily injection of 10 μg GM-CSF-EGF fusion protein of Example 1 for 10 days (the study group). Twenty-one days after the tumor inoculation, the mice underwent in vivo imaging to detect the bioluminescent intensity (a marker of tumor burden) resulted from luciferase expressed by RENCA-hEGFR-luc cells. As show in FIGS. 6A and 6B, the control group displayed a significant higher bioluminescent intensity (3 folds higher) resulted from RENCA-hEGFR-luc cells compared to that of the study group.

The results suggest the mGM-CSF-hEGF and the mGM-CSF-hVEGF fusion proteins are effective in inhibiting cancer cells.

### Example 4

### The Binding of mGM-CSF-hEGF to EGFR on Cancer Cells

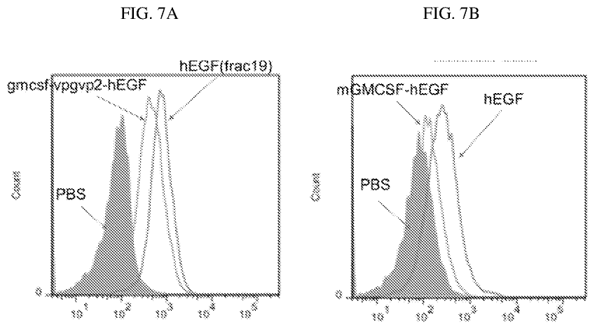

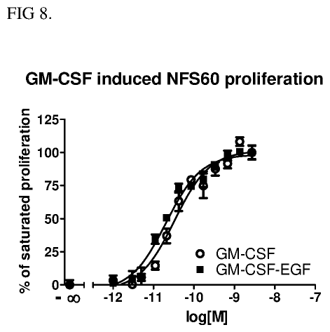

An in vitro study evaluating the binding of mGM-CSF-hEGF to cancer cells expressing human EGFR, including RENCA-hEGFR-luc stable cells and MDA-468 breast cancer cells, was performed. The EGFR expressed on MDA-468 breast cancer cells and RENCA-hEGFR-luc cells could bind to purified his6-tagged hEGF and mGM-CSF-hEGF. The binding of hEGF and mGM-CSF-hEGF to the EGFR was detected using FITC-labeled antibody specific for his6-tag and FACS analysis. The fluorescent intensities of FIG. 7A and FIG. 7B show represent the amount of his6-tagged hEGF (bright blue) or his6-tagged mGM-CSF-hEGF (orange) coupled to EGFR.

The FACS images show the binding of GM-CSF-EGF conjugate of Example 1 to cancer cells expressing human EGFR. This result indicates the GM-CSF-EGF bound cancer cells may be used as a cancer vaccine.

### Example 5

### The Immunogenicity of GM-CSF-EGF Fusion Protein

An in vivo study was conducted to evaluate the immunogenicity of GM-CSF-EGF fusion protein using balb/c mice.

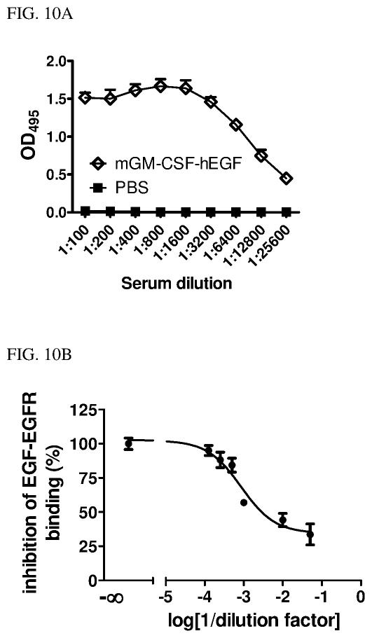

Each Balb/c and c57/BL mouse was subcutaneously injected with 10 μg of GM-CSF-EGF fusion protein of Example 1 (SEQ ID NO:1) or Phosphate-buffered saline (PBS) daily for 2 weeks. Serum anti-EGF antibody was measured 4 weeks after the last injection. FIG. 10A shows the anti-EGF antibody titer in the serum of mice injected with GM-CSF-EGF fusion protein was as high as 25,600, four weeks after the conjugate administration. No discernible side effect, such as weight loss, ruffled fur, or any sign of distress, was noted in the mice. FIG. 10B shows the serum of mice injected with mGM-CSF-EGF fusion protein suppresses the binding of EGF to EGFR.

The serums were examined for their ability to block association between EGF and EGFR. Purified EGFR was immobilized on the 96-well ELISA plate and incubated with serially diluted serum from mice immunized with either GM-CSF-EGF fusion protein or PBS. Similar to the previously described in vitro EGF-EGFR binding assay, the EGFR coupled his6-tagged EGF was measured using a HRP-conjugated anti-his6-tag antibody in the presence of HRP substrate (100 μL/well), 3,30,5,50-tetramethylbenzidine (TMB). The peroxidase reaction was stopped 30 min after the addition of 0.5 M H2SO4 (50 mL/well), and the absorbance was measured at 450 nm with a multichannel microtiter plate reader. The EGFR-EGF binding in the absence of serum was determined as 100%. Increasing concentration of serum from GM-CSF-EGF immunized mice, ranging from dilution of 4000 to 20 folds, suppressed the association between EGF and EGFR.

### Example 6

### The synergistic Anti-cancer Efficacy of mGM-CSF-hEGF Fusion Protein and Anti-CTLA4 Antibody (9H10) Combinations

An in vivo study was conducted to evaluate the anticancer efficacy of mGM-CSF-hEGF fusion protein (SEQ ID NO:1) combined with an anti-CTLA4 antibody (9H10) using c57 mice.

The mice were divided into 4 groups and subcutaneously inoculated with the following: Group A mice with 1×10⁵ B16-F10 murine melanoma cells expressing hEGFR and 1×10⁵ B16-F10 murine melanoma cells expressing mGM-CSF (B16F10 hEGFR/B16mGMCSF), group B with 1×10⁵ B16-F10 murine melanoma cells expressing EGFR and 1×10⁵ stable B16-F10 murine melanoma cells expressing mGM-CSF-hEGF (B16F10 hEGFR/B16mGMCSF-hE), Group C mice with 1×10⁵ stable B16-F10 murine melanoma cells expressing hEGFR mixed with 1×10⁵ stable B16-F10 murine melanoma cells expressing mGM-CSF combined with 9H10 antibody (B16F10 hEGFR/B16F10 mG/9H10) and group D mice with 1×10⁵ stable B16-F10 murine melanoma cells expressing hEGFR mixed with 1×10⁵ stable B16-F10 murine melanoma cells expressing mGM-CSF-hEGF combined with 9H10 antibody (B16F10 hEGFR/B16F10 mG-hE/9H10).

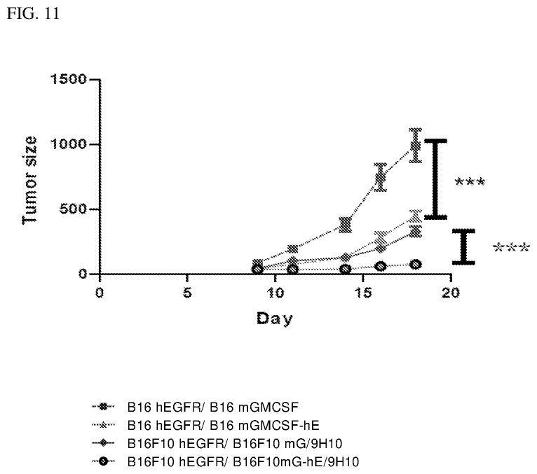

FIG. 11 shows the tumor size in the 4 groups of mice inoculated with the B16-F10 cells expressing mGM-CSF or mGM-CSF-hEGF, with or without anti CTLA-4 antibody. Group C and Group D mice had significant tumor reduction compared to group A mice (inoculated with cells expressing mGM-CSF) or group B mice (inoculated with cells expressing mGM-CSF-hEGF). Group D mice (inoculated with cells expressing mGM-CSF-hEGF combined with 9H10 antibody) had significant tumor reduction compared to group C mice (inoculated with cells expressing mGM-CSF combined with 9H10 antibody).

The results suggest the combination of mGM-CSF-hEGF with an anti CTLA-4 antibody is synergistic in cancer cell inhibition.

### Example 7

### The Anti-cancer Efficacy of the GM-CSF-PDGFA and GM-CSF-bFGF Fusion Proteins

An in vivo study was conducted to evaluate the anticancer efficacy of GM-CSF-PDGFA (SEQ ID NO:3) and GM-CSF- bFGF (SEQ ID NO:4) fusion proteins using c57 mice.

The mice were subcutaneously inoculated with (a) 5×10⁴ stable B16-F10 murine melanoma cells expressing mGM-CSF, (b) 0.75×10⁴ parental B16-F10 murine melanoma cells mixed with 4.25×10⁴ stable B16-F10 murine melanoma cells expressing mGM-CSF-mPDGFA or (c) 2.7×10⁴ parental B16-F10 murine melanoma cells mixed with 2.3×10⁴ stable B16-F10 murine melanoma cells expressing mGM-CSF-mbFGF.

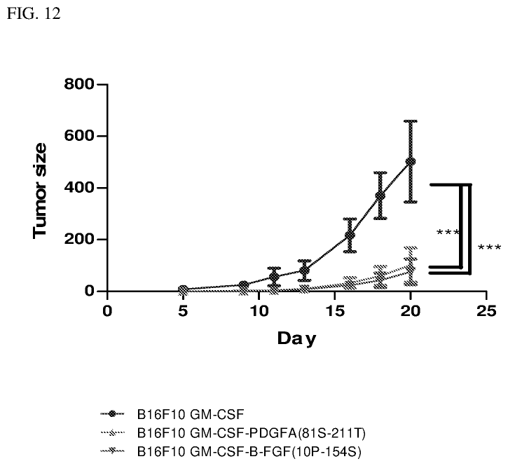

Referring to FIG. 12, mice inoculated with the B16-F10 cells expressing mGM-CSF-mPDGFA (group b) and mGM-CSF-mbFGF (group c) had significant tumor reduction compared to the mice inoculated with B16-F10 cells tumor expressing mGM-CSF only (group a).

The results demonstrate that mGM-CSF-mPDGFA and the mGM-CSF-mbFGF fusion proteins are effective in inhibiting cancer cells.

While certain embodiments of the invention have been illustrated and described, those skilled in the art with access to the present teachings will recognize that the invention is not limited to these embodiments only. Accordingly, it is to be understood that the invention is intended to cover numerous modifications, changes, variations, substitutions, and equivalents that will be apparent to those skilled in the art.

What is claimed is:

1. A conjugate, comprising
a granulocyte macrophage colony stimulating factor (GM-CSF) polypeptide; and
a ligand.

2. The conjugate of claim 1, wherein the GM-CSF is operatively linked to the ligand by a linker or covalently linked to the ligand.

3. The conjugate of claim 1, wherein the ligand is a tumor associated ligand.

4. The conjugate of claim 3, wherein the tumor associate ligand is selected from epidermal growth factor (EGF), C-X-C motif chemokine 12 (CXCL12), Hepatocyte Growth Factor-1 (HGF) and Insulin-like Growth Factor (IGF).

5. The conjugate of claim 3, wherein the tumor associated ligand is selected from colony stimulating factor (CSF-1), macrophage chemoattractant protein-I (MCP-1) and macrophage inflammatory protein-1α (MIP-1α).

6. The conjugate of claim 3, wherein the tumor associated ligand is selected from vascular endothelial growth factor (VEGF), Platelet Derived Growth Factor (PDGF) and Fibroblast growth factor (FGF).

7. The conjugate of claim 1, wherein the ligand is not interleukin, an antibody or a receptor on a tumor cell.

8. The conjugate of claim 1, wherein the conjugate is at least 90% homologous to SEQ ID NO: 1, SEQ ID NO: 2, SEQ ID NO:3 or SEQ ID NO:4.

9. A pharmaceutical composition, comprising
the conjugate of claim 1; and
a pharmaceutically acceptable excipient.

10. The pharmaceutical composition of claim 9, further comprises an anti-cancer agent.

11. The pharmaceutical composition of claim 10, wherein the anti-cancer agent is selected from a Cytotoxic T-lymphocyte-associated antigen 4 (CTLA-4) antibody, an EGFR antibody, or a tyrosine kinase inhibitor.

12. A method for inhibiting cancer cell growth in a subject, comprising administering an effective amount of a conjugate comprising a GM-CSF and a ligand, wherein said effective amount of the conjugate inhibit cancer cell in said subject.

13. The method of claim 12, wherein the GM-CSF is covalently or operatively linked to the ligand.

14. The method of claim 12, wherein the ligand is a tumor associated ligand.

15. The method of claim 14, wherein the tumor associate ligand is selected from epidermal growth factor (EGF), C-X-C motif chemokine 12 (CXCL12), Hepatocyte Growth Factor-1 (HGF) and Insulin-like Growth Factor (IGF), colony stimulating factor (CSF-1), macrophage chemoattractant protein-I (MCP-1) and macrophage inflammatory protein-1α (MIP-1α), vascular endothelial growth factor (VEGF), Platelet Derived Growth Factor (PDGF) or Fibroblast growth factor (FGF).

16. The method of claim 12, wherein the conjugate is at least 90% homologous to SEQ ID NO: 1, SEQ ID NO: 2, SEQ ID NO:3 or SEQ ID NO:4.

17. The method of claim 12, further comprising administering an effective amount of an anti-cancer agent.

18. The method of claim 17, wherein the anti-cancer agent is selected from an CTLA-4 antibody, an EGFR antibody, or a tyrosine kinase inhibitor.

19. The method of claim 12, wherein the cancer cell expresses epidermal growth factor receptor (EGFR) or vascular endothelial growth factor (VEGF).

20. A method for inducing anti-EGF antibody in a subject, comprising administering an effective amount of a conjugate comprising GM-CSF and EGF in the subject in need of cancer treatment, wherein an effective amount of anti-EGF antibody is induced in said subject.

21. A method for inducing anti-VEGF antibody in a subject, comprising administering an effective amount of a conjugate comprising GM-CSF and VEGF in the subject in need of cancer treatment, wherein an effective amount of anti-VEGF antibody is induced in said subject.
